SEQUENCE LISTING (29 sequences)

SEQ ID NO: 1 - protein, 16 aa, synthetic construct
  SITE 1: note = d-Lysine
  REGION 1..16: note = Peptidomimetic of 32_33 peptide All amino acids are d aminoacids
  SITE 2: note = d-Lysine
  SITE 3: note = d-Serine
  SITE 4: note = d-Phenylalanine
  SITE 5: note = d-Leucine
  SITE 6: note = d-Serine
  SITE 7: note = d-Proline
  SITE 8: note = d-Arginine
  SITE 9: note = d-Threonine
  SITE 10: note = d-Alanine
  SITE 11: note = d-Leucine
  SITE 12: note = d-Isoleucine
  SITE 13: note = d-Asparagine
  SITE 14: note = d-Phenylalanine
  SITE 15: note = d-Leucine
  SITE 16: note = d-Valine
KKSFLSPRTALINFLV

SEQ ID NO: 2 - protein, 8 aa, synthetic construct
  SITE 1: note = d-Lysine
  REGION 1..8: note = all amino acids are d amino acids
  SITE 2: note = d-Lysine
  SITE 3: note = d-Serine
  SITE 4: note = d-Phenylalanine
  SITE 5: note = d-Leucine
  SITE 6: note = d-Serine
  SITE 7: note = d-Proline
  SITE 8: note = d-Arginine
KKSFLSPR

SEQ ID NO: 3 - protein, 8 aa, synthetic construct
  SITE 1: note = d-Leucine
  REGION 1..8: note = all amino acids are d amino acids
  SITE 2: note = d-Serine
  SITE 3: note = d-Proline
  SITE 4: note = d-Arginine
  SITE 5: note = d-Threonine
  SITE 6: note = d-Alanine
  SITE 7: note = d-Leucine
  SITE 8: note = d-Isoleucine
LSPRTALI

SEQ ID NO: 4 - protein, 16 aa, synthetic construct
  REGION 1..16: note = As published by Perli et al, FASEB J, 2020
KKSFLSPRTALINFLV

SEQ ID NO: 5 - protein, 20 aa, synthetic construct
  SITE 1: note = d-Phenylalanine
  SITE 2: note = d-Arginine
  SITE 3: note = d-Phenylalanine
  SITE 4: note = d-Lysine
  SITE 5: note = d-Lysine
  SITE 6: note = d-Lysine
  SITE 7: note = d-Serine
  SITE 8: note = d-Phenylalanine
  SITE 9: note = d-Leucine
  SITE 10: note = d-Serine
  SITE 11: note = d-Proline
  SITE 12: note = d-Arginine
  SITE 13: note = d-Threonine
  SITE 14: note = d-Alanine
  SITE 15: note = d-Leucine
  SITE 16: note = d-Isoleucine
  SITE 17: note = d-Asparagine
  SITE 18: note = d-Phenylalanine
  SITE 19: note = d-Leucine
  SITE 20: note = d-Valine
  REGION 1..20: note = all amino acids are d amino acids
FRFKKKSFLSPRTALINFLV

SEQ ID NO: 6 - protein, 12 aa, synthetic construct
  SITE 1: note = d-Phenylalanine
  SITE 2: note = d-Arginine
  SITE 3: note = d-Phenylalanine
  SITE 4: note = d-Lysine
  SITE 5: note = d-Lysine
  SITE 6: note = d-Lysine
  SITE 7: note = d-Serine
  SITE 8: note = d-Phenylalanine
  SITE 9: note = d-Leucine
  SITE 10: note = d-Serine
  SITE 11: note = d-Proline
  SITE 12: note = d-Arginine
  REGION 1..12: note = all amino acids are d amino acids
FRFKKKSFLSPR

SEQ ID NO: 7 - protein, 12 aa, synthetic construct
  SITE 1: note = d-Phenylalanine
  SITE 2: note = d-Arginine
  SITE 3: note = d-Phenylalanine
  SITE 4: note = d-Lysine
  SITE 5: note = d-Lysine
  SITE 6: note = d-Serine
  SITE 7: note = d-Proline
  SITE 8: note = d-Arginine
  SITE 9: note = d-Threonine
  SITE 10: note = d-Alanine
  SITE 11: note = d-Leucine
  SITE 12: note = d-Isoleucine
  REGION 1..12: note = all amino acids are d amino acids
FRFKLSPRTALI

SEQ ID NO: 8 - protein, 4 aa, synthetic construct
  REGION 1..4
  SITE 1: note = d-Phenylalanine
  SITE 2: note = d-Arginine
  SITE 3: note = d-Phenylalanine
  SITE 4: note = d-Lysine
  REGION 1..4: note = all amino acids are d-amino acids
FRFK

SEQ ID NO: 9 - protein, 4 aa, synthetic construct
  SITE 1: note = d-Phenylalanine
  SITE 2: note = d-Arginine
  SITE 3: note = d-cyclohexylalanine
  SITE 4: note = d-Lysine
FRAK

SEQ ID NO: 10 - protein, 4 aa, synthetic construct
  SITE 2: note = d-Arginine
FRFK

SEQ ID NO: 11 - protein, 4 aa, synthetic construct
  SITE 1: note = d-cyclohexylalanine
  SITE 2: note = d-Arginine
  SITE 3: note = d-cyclohexylalanine
  SITE 4: note = d-Lysine
ARAK

SEQ ID NO: 12 - protein, 8 aa, synthetic construct
  SITE 1: note = d-Phenylalanine
  SITE 2: note = d-Arginine
  SITE 3: note = d-Phenylalanine
  SITE 4: note = d-Lysine
  SITE 5: note = d-Phenylalanine
  SITE 6: note = d-Arginine
  SITE 7: note = d-Phenylalanine
  SITE 8: note = d-Lysine
FRFKFRFK

SEQ ID NO: 13 - protein, 8 aa, synthetic construct
  SITE 1: note = d-Phenylalanine
  SITE 2: note = d-Arginine
  SITE 3: note = d-cyclohexylalanine
  SITE 4: note = d-Lysine
  SITE 5: note = d-Phenylalanine
  SITE 6: note = d-Arginine
  SITE 7: note = d-cyclohexylalanine
  SITE 8: note = d-Lysine
FRAKFRAK

SEQ ID NO: 14 - protein, 8 aa, synthetic construct
  SITE 1: note = d-cyclohexylalnine
  SITE 2: note = d-Arginine
  SITE 3: note = d-cyclohexylalanine
  SITE 4: note = d-Lysine
  SITE 5: note = d-cyclohexylalanine
  SITE 6: note = d-Arginine
  SITE 7: note = d-cyclohexylalanine
  SITE 8: note = d-Lysine
ARAKARAK

SEQ ID NO: 15 - protein, 9 aa, synthetic construct
  SITE 1: note = d-Arginine
  SITE 2: note = d-Lysine
  SITE 3: note = d-Lysine
  SITE 4: note = d-Arginine
  SITE 5: note = d-Arginine
  SITE 6: note = d-Glutamine
  SITE 7: note = d-Arginine
  SITE 8: note = d-Arginine
  SITE 9: note = d-Arginine
RKKRRQRRR

SEQ ID NO: 16 - protein, 4 aa, synthetic construct
  SITE 1: note = d-Phenylalanine
  SITE 2: note = d-Arginine
  SITE 3: note = d-diphenylalanine
  SITE 4: note = d-Lysine
FRFK

SEQ ID NO: 17 - protein, 4 aa, synthetic construct
  SITE 1: note = d-Phenylalanine
  SITE 2: note = d-Arginine
  SITE 3: note = d-methylated tyrosine
  SITE 4: note = d-Lysine
FRYK

SEQ ID NO: 18 - protein, 4 aa, synthetic construct
  SITE 1: note = d-Phenylalanine
  SITE 2: note = d-Arginine
  SITE 3: note = d-Tyrosine
  SITE 4: note = d-Lysine
FRYK

SEQ ID NO: 19 - protein, 4 aa, synthetic construct
  SITE 1: note = d-Tyrosine
  SITE 2: note = d-Arginine
  SITE 3: note = d-Tyrosine
  SITE 4: note = d-Lysine
YRYK

SEQ ID NO: 20 - protein, 4 aa, synthetic construct
  SITE 2: note = d-Arginine
  SITE 3: note = cyclohexylalanine
FRAK

SEQ ID NO: 21 - protein, 4 aa, synthetic construct
  SITE 1: note = cyclohexylalanine
  SITE 2: note = d-Arginine
  SITE 3: note = cyclohexylalanine
ARAK

SEQ ID NO: 22 - protein, 8 aa, synthetic construct
  SITE 2: note = d-Arginine
  SITE 6: note = d-Arginine
FRFKFRFK

SEQ ID NO: 23 - protein, 8 aa, synthetic construct
  SITE 2: note = d-Arginine
  SITE 3: note = cyclohexylalanine
  SITE 6: note = d-Arginine
  SITE 7: note = cyclohexylalanine
FRAKFRAK

SEQ ID NO: 24 - protein, 8 aa, synthetic construct
  SITE 1: note = cyclohexylalanine
  SITE 2: note = d-Arginine
  SITE 3: note = cyclohexylalanine
  SITE 5: note = cyclohexylalanine
  SITE 6: note = d-Arginine
  SITE 7: note = cyclohexylalanine
ARAKARAK

SEQ ID NO: 25 - protein, 9 aa, synthetic construct
RKKRRQRRR

SEQ ID NO: 26 - protein, 4 aa, synthetic construct
  SITE 2: note = d-Arginine
  SITE 3: note = diphenylanine
FRFK

SEQ ID NO: 27 - protein, 4 aa, synthetic construct
  SITE 2: note = d-Arginine
  SITE 3: note = methylated tyrosine
FRYK

SEQ ID NO: 28 - protein, 4 aa, synthetic construct
  SITE 2: note = d-Arginine
FRYK

SEQ ID NO: 29 - protein, 4 aa, synthetic construct
  SITE 2: note = d-Arginine
YRYK

(19) United States
(12) Patent Application Publication
(10) Pub. No.: US 2025/0171762 A1
(43) Pub. Date: May 29, 2025

(54) THERAPEUTICAL PEPTIDOMIMETIC

(71) Applicants: UNIVERSITA' DEGLI STUDI DI ROMA "LA SAPIENZA", Roma (IT); CONSIGLIO NAZIONALE DELLE RICERCHE, Roma (IT); ASSOCIATION FRANÇAISE CONTRE LES MYOPATHIES – AFM-TÉLÉTHON, Evry (FR)

(72) Inventors: Giulia D'AMATI, Roma (IT); Veronica MOREA, Roma (IT); Annalinda PISANO, Roma (IT); Elena PERLI, Roma (IT); Maria Gemma PIGNATARO, Roma (IT)

(73) Assignees: UNIVERSITA' DEGLI STUDI DI ROMA "LA SAPIENZA", Roma, RM (IT); CONSIGLIO NAZIONALE DELLE RICERCHE, Roma, RM (IT); ASSOCIATION FRANÇAISE CONTRE LES MYOPATHIES – AFM-TÉLÉTHON, Evry (FR)

(21) Appl. No.: 18/725,699

(22) PCT Filed: Dec. 16, 2022
(86) PCT No.: PCT/IB2022/062354

§ 371 Date: Jun. 28, 2024

(30) Foreign Application Priority Data
Dec. 29, 2021 (IT) 102021000032930

(51) Int. Cl. C12N 9/00; A61K 38/53

(52) U.S. Cl. CPC C12N 9/93; A61K 38/53; C12Y 601/01004

(57) ABSTRACT

The present invention relates to peptidomimetics of a short peptide from leucyl-tRNA synthetase, compositions comprising one or more of said peptidomimetics and their use for the treatment of syndromes caused by mutations of mt-tRNA (mitochondrial transfer RNA) genes, and medical treatments of said syndromes comprising the administration of said one or more peptidomimetics or compositions comprising the same.

The present invention relates to peptidomimetics of a short peptide from leucyl-tRNA synthetase, compositions comprising one or more of said peptidomimetics and their use for the treatment of syndromes caused by mutations of mt-tRNA (mitochondrial transfer RNA) genes, and medical treatments of said syndromes comprising the administration of said one or more peptidomimetics or compositions comprising them.

### STATE OF THE ART

Mitochondrial (mt) diseases due to mutations in transfer RNA (tRNA) genes are responsible for a wide range of syndromes, for which no effective treatment is available at present. Mitochondrial tRNA (mt-tRNA) genes are “hotspots” for pathological mutations and over 200 mt-tRNA mutations have been linked to various disease states. Often these mutations prevent tRNA aminoacylation. It is believed that disrupting this primary function affects protein synthesis and the expression, folding, and function of oxidative phosphorylation enzymes. Mitochondrial tRNA mutations manifest in a wide panoply of diseases related to cellular energetics, including mitochondrial myopathy, MERRF (Myoclonic Epilepsy with Ragged Red Fibers), MIDD (Maternally Inherited Diabetes and Deafness) and MELAS (mitochondrial encephalomyopathy, lactic acidosis, and stroke-like episodes). Diseases caused by mt-tRNA mutations can also affect very specific tissue types, as in the case of neurosensory non-syndromic hearing loss and pigmentary retinopathy, diabetes mellitus, and hypertrophic cardiomyopathy. In particular, mutations in mitochondrial genes coding for mt-tRNAs, such as m·3243A>G in MT-TL1 human gene encoding mt-tRNA^{Leu(UUR)} and m·8344A>G in MT-TK human gene encoding mt-tRNA^{Lys}, cause an impairment in the mt tRNA structure resulting in impaired tRNA interactions with aminoacyl-tRNA synthetases and other molecules (such as proteins, mRNA, ribosomes) thereby leading to impaired tRNA physiological functions.

The two mutations above are responsible, together, for the most common and severe human mt-tRNA-related diseases (about 85%). In particular, mitochondrial tRNA mutation m·3243A>G in MT-TL1 human gene encoding mt-tRNA^{Leu(UUR)} is known to cause MELAS (Mitochondrial Encephalopathy, Lactic Acidosis and Stroke-like episodes) and MIDD (Maternally Inherited Diabetes and Deafness) whereas mutation m·8344A>G in MT-TK human gene encoding mt-tRNA^{Lys} is known to cause MERRF (Myoclonic Epilepsy with Ragged Red Fibers).

Said diseases normally show their onset in adolescence and early adulthood and affect the many organs and tissues requiring high energy, such as CNS, Heart, Skeletal muscle) causing a number of symptoms. The symptoms shown by patients affected by MELAS include seizures, dementia, stroke-like episodes, muscular weakness, hypoacusia, heart conduction problems; the symptoms shown by patients affected by MIDD include diabetes and deafness and the symptoms shown by patients affected by MERRF include ataxia, myoclonus, muscular atrophy and dementia.

The clinical course of said diseases is chronic, progressive and ultimately fatal.

A number of diverse approaches has been explored so far to counteract the symptoms of these diseases, including DNA manipulation, protein delivery and development of small molecule pharmaceuticals. At present, the available treatments are poorly effective and include generic enhancers of mt function such as B group vitamins (cofactors of enzymes catalysing essential reactions); E vitamin and Coenzyme Q10 (antioxidants); amino acids and other nutrient supplements that are empirically administered as «cocktails» to patients, based on biochemical reasoning and consensus expert opinion. No current treatment is able to effectively manage the symptoms nor to specifically address the underlying mechanisms of these syndromes, i.e., the mutated tRNA instability.

Among the tRNA targeted therapies, overexpression of human cognate or non-cognate aminoacyl mt-tRNA synthetases has been proven to rescue defective phenotypes in human trans-mt hybrids (cybrids) a well-established cellular model of mt-tRNA mutations.

Perli et al demonstrated (Perli et al EMBO Molecular Medicine 2014, Vol 6 No 2 169-182) that plasmids encoding for the non-catalytic carboxy-terminal domain (Cterm) of human mt-LeuRS (67-residues long), joined or not with a well characterized mt targeting sequence (from either the Neurospora crassa F0-ATPase subunit 9 precursor or human COX8a), effectively rescue the defects of human cybrids carrying the mutation m·3243A>G in mt-tRNA^{Leu(UUR)} or m·8344A>G in mt-tRNA^{Lys}. Subsequently, in Perli et al 2016 (Perli et al “short peptides from leucyl-tRNA synthetase rescue disease-causing mitochondrial tRNA point mutations” Hum mol genet 2016, Vol 25 No 5 903-915) the authors demonstrated that transfection of plasmids encoding for the short Cterm-derived β32_33 (16-residues long) or β30_31 (15-residues long) sequence joined with the COX8a mt targeting sequence, have the same rescuing abilities as the Cterm on both mutant cybrids. The authors also showed that β32_33 is endowed with higher rescuing activity than both β30_31 and Cterm. Finally, the authors highlighted by in vitro experiments that the β32_33 and β30_31 peptides are able to strongly interact with, and stabilize a functional conformation of mutant mt-tRNA^{Leu(UUR)} or mt-tRNA^{Lys}. Direct in vitro interaction with, and stabilization of target tRNAs suggested that the rescuing effect was mediated by a “chaperonic” activity.

More recently, in Perli et al 2020 (Perli et al, FASEB J, Vol 34 No 6 7675-7686) the authors demonstrated that the exogenously administered β32_33 peptide is able per se to penetrate both the plasma and mitochondrial membranes and exert the rescuing activity on mutant cells.

Importantly, the observation that the rescuing effect of the mt-LeuRS-derived sequences is exerted not only on the cognate mt-tRNA^{Leu(UUR)} but also on the non-cognate mt-tRNA^{Lys}, led the authors to propose that these sequences may be active on a large spectrum of human mt-tRNA mutants. Indeed, transfection of plasmids encoding for either β32_33 or β30_31 sequences has been demonstrated to rescue the phenotype of a large panel of mt-tRNA mutants in the yeast model (Di Micco, P. et al 2014 “The yeast N-model suggests the use of short peptides derived from mt LeuRS for the therapy of diseases due to mutations in several mt tRNAs”. Biochim. Biophys. Acta, 1843, 3065-3074).

Conjugation with a mitochondrial penetrating sequence (herein disclosed as SEQ ID NO 10) did not increase rescuing activity or mitochondrial localization, indicating that the β32_33 peptide is endowed per se with mitochondrial targeting properties (Perli et al 2020, “Exogenous peptides are able to stabilize mitochondrial tRNAs, penetrate human cell and mitochondrial membranes and rescue severe mitochondrial defects” FASEB J Vol 34 No 6 7675-7686). In the paper the authors demonstrated that constructs where the β32_33 peptide sequence is scrambled or positively charged residues are mutated to alanine, do not possess rescuing activity, and indicated that both the order of amino acids along the sequence and the presence of positive charges are essential determinants of the peptide effect. The β32_33 peptide is therefore a promising molecule for the development of therapeutic agents against diseases caused by mt-tRNA point mutations. The authors also indicate that the β32_33 peptide is a promising lead molecule for the development of non-peptide derivatives endowed with rescuing activity toward mutations in mt-tRNAs.

Notwithstanding the promising data on possible therapeutic peptides, there is a need to provide molecules that not only show a rescuing activity towards mutations in mt-tRNAs but that are also suitable to be used in therapy, hence there is a strong clinical need for the identification of molecules suitable for the treatment mt-tRNA-related diseases such as MELAS, MIDD and MERRF, as well as other mt diseases caused by mt-tRNA point mutations.

### SUMMARY OF THE INVENTION

The results reported in Perli et al 2020, indicate that the β32_33 peptide is a promising lead molecule for the development of non-peptide derivatives endowed with rescuing activity toward mutations in mt-tRNAs. However, the strong degradation generally observed with peptide molecules renders the peptide itself unsuitable for use in therapy unless its stability in blood can be enhanced.

Perli et al 2020, provides clear information on the essential features responsible for β32_33 peptide rescuing activity, which can be exploited for the development of therapeutic agents against human mt-tRNA mutation related diseases. These features are the spatial arrangement of residues and number of positive charges.

In other words, the spatial arrangement of residues and number of positive charges are indicated as essential features of β32_33 peptide rescuing activity that the skilled person should take into account when designing possible peptidomimetics of said peptide. The meaning of this sentence is that, according to the state of the art, it is necessary for the β32_33 peptide (βp) to possess both features to exert high rescuing activity.

This statement is supported by the results presented in the same paper where two peptides derived from β32_33 and differing in only one of the two features (respectively spatial arrangement of residues and number of positive charges), as reported in Table 1 of the paper, one obtained by scrambling (i.e. same amino acid residues but in a different order within the peptide) and the other one by substitution of three positively charged amino acids K1, K2 and R8 (numbers indicate the position of the amino acid along the peptide side-chain), show a significantly lower rescuing activity with respect the β32_33peptide.

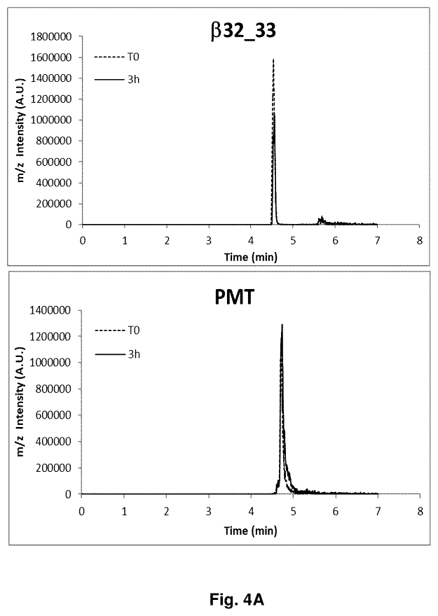

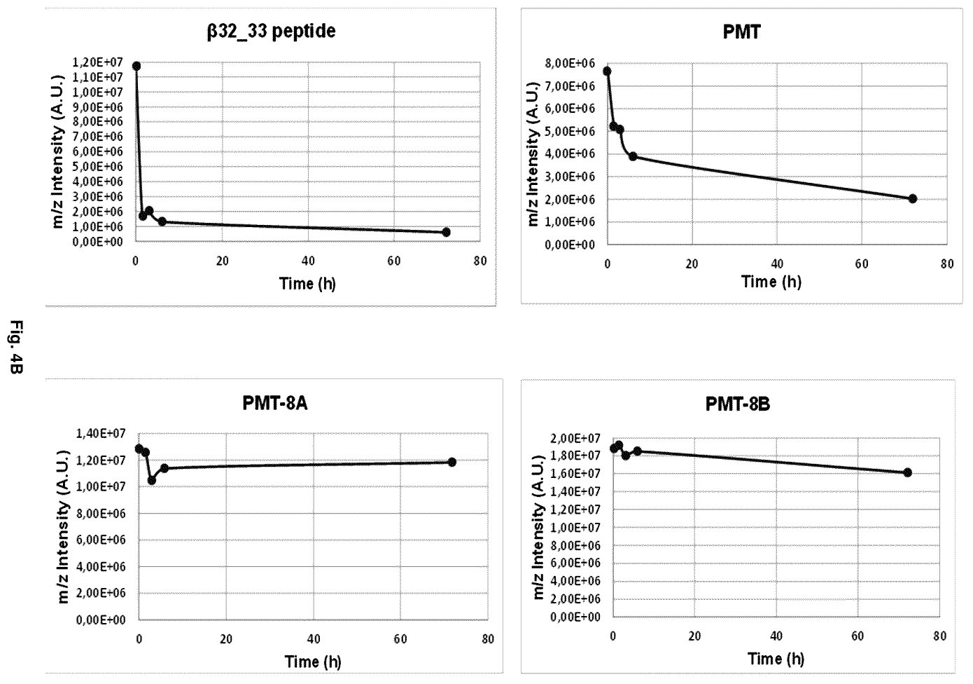

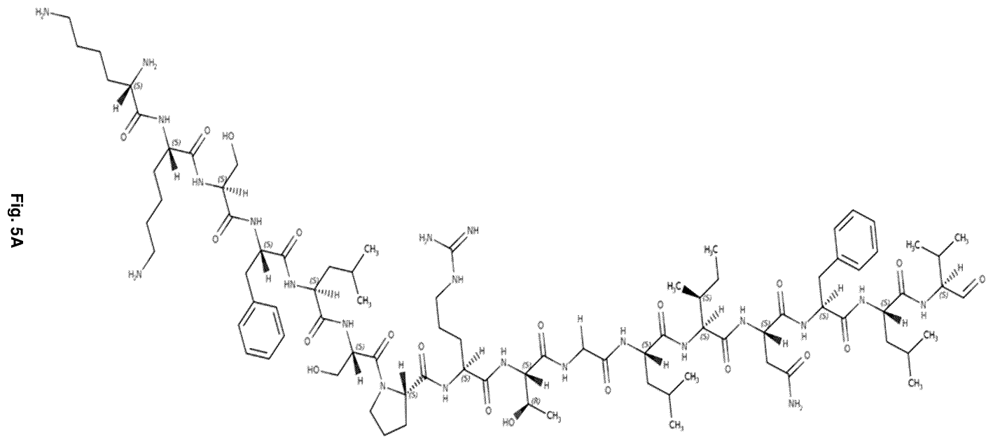

The present inventors have verified that the β32_33 peptide disclosed in Perli et al 2016 and Perli et al 2020 (SEQ ID NO 4), has a short stability in blood as shown in FIG. 4. Panel A of FIG. 4 shows that, in a first experiment, the β32_33 prior art peptide (SEQ ID NO 4) is significantly degraded (at least 30% of degraded peptide is observed) after three hours incubation in plasma of two healthy volunteers at 37° C., i.e., human body temperature. Panel B of FIG. 4 shows that, in a second experiment, the β32_33 peptide is degraded to an even larger extent, since more than 85% of the starting amount is present after 1.5 h incubation in plasma of four healthy volunteers, different from the two of the previous experiment. In spite of the different degradation rate shown by the β32_33 peptide and PMT (SEQ ID NO 1) in the two experiments, where plasma samples from different volunteers have been used, the PMT shows a consistently higher stability than the β32_33 peptide, since after 3 hours incubation in this medium, in a first experiment the PMT is 100% available vs. 70% of the β32_33 peptide (FIG. 4A), and in a second experiment the PMT is >63% available vs. only 17% of the β32_33 peptide (FIG. 4B). Interestingly, as shown in FIG. 4, panel B, the PMT smaller fragments PMT-8a and PMT-8b (respectively, SEQ ID 2 and 3), both of which are 8-residue long, are even more stable than the full-length 16-residue long PMT. This result suggests that the plasma stability of the PMT may be further increased by ad hoc chemical modifications affecting the 4^{th} (i.e., d-Phe), 5^{th} (i.e., d-Leu), 8^{th} (i.e., d-Arg) and/or 9^{th} (i.e., d-Thr) residue of the PMT, and/or the peptide bond between the 4^{th} and 5^{th} residues (i.e., d-Phe and d-Leu) and/or between the 8^{th} and 9^{th} residue (i.e., d-Arg and d-Thr). Additionally, plasma stability of the PMT may be further increased by modifications affecting additional PMT residues the peptide bond between which may be putatively cleaved in human plasma. The poor stability of the prior art β32_33 peptide disclosed in Perli 2016 was expected since this peptide is produced by transfection in the cells and, therefore, consists of I-amino acids (the same applies to Perli 2020 where the sole d-amino acid disclosed in the paper is the d-Arg amino acid of the short peptide of four amino acids for mt-targeting disclosed in the paper) and does not have further modifications that could enhance its stability.

The poor in vivo stability of unmodified peptides (such as β32_33 of SEQ ID NO 4), against proteolysis is, in fact, a major challenge that must be overcome, as it can result in an impractically short in vivo biological half-life and a subsequently poor bioavailability when used in imaging and therapeutic applications. Because of poor stability, many biologically and pharmacologically interesting peptide-based drugs may never see application. The skilled person knows that a potential way to overcome this limitation is the use of peptide analogues designed to mimic the pharmacophore of a native peptide while also containing unnatural modifications that act to maintain or improve the pharmacological properties. Various strategies have been developed to increase the metabolic stability of peptide-based pharmaceuticals. These include modifications of the C- and/or N-termini, introduction of d-or other unnatural amino acids, backbone modification, PEGylation and alkyl chain incorporation, cyclization and peptide bond substitution, and all said strategies have been, or could be, applied to peptide-based pharmaceuticals.

Although the use of d-amino acids is known in the art, the skilled person also knows (e.g., Evans et al Molecules. 2020 May; 25 (10): 2314 Methods to Enhance the Metabolic Stability of Peptide-Based PET Radiopharmaceuticals) that the simple substitution of all I-amino acids in a peptide with d-amino acids is generally an ineffective strategy as the resulting changes in peptide conformation and side chain orientation can prevent the correct binding geometry and thus destroy target binding. Additionally, peptide bond cleavage by plasma or liver hydrolases is only one of the possible reasons underlying the in vivo short-life of peptide compounds, which can also undergo elimination through the kidneys and/or sequestration by plasma or tissue proteins. The main example of such proteins is serum albumin, which is able to bind molecules comprising hydrophobic regions and carry them to the liver for degradation, thus reducing the amount of such molecules that is free in the bloodstream and free to diffuse to cells and tissues.

For this reason, the benefits offered by d-amino acids to a peptide are normally sought without substituting every amino acid with its d-amino acid equivalent. For example, substituting the N-terminal I-amino acid of most proteins with the corresponding d-amino acid can significantly increase in vivo stability by preventing recognition of the N-terminus of the protein by proteases.

In addition, Perli et al 2020 clearly teaches that spatial arrangement of residues and number of positive charges are essential features for the rescuing activity of the β32_33 peptide of SEQ ID NO 4, therefore the inventors were, indeed, surprised, to find that a peptide having the same sequence of the β32_33 peptide in which all I-amino acids were substituted with d-amino acids, hence, not maintaining the above-mentioned essential features,

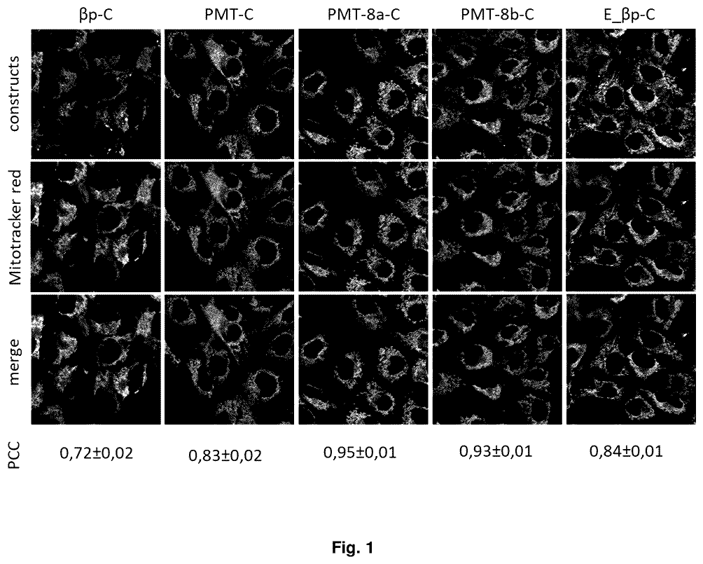

maintained the same ability to penetrate cell and mt membranes upon exogenous administration (FIG. 1) of the natural β32_33 peptide,

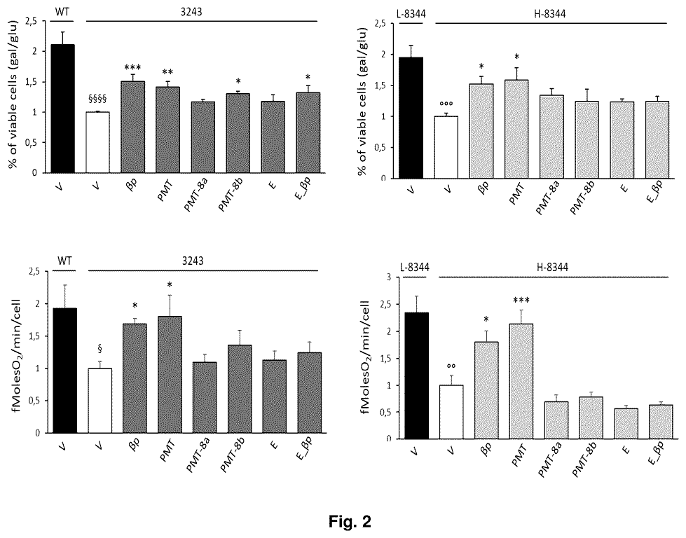

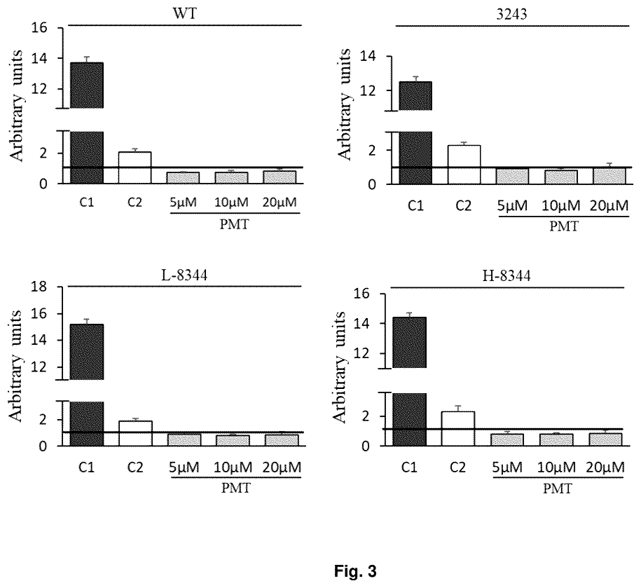

maintained the same rescuing of the defective phenotype of cell models carrying the m·3243A>G mutation in the gene coding for mt-tRNA^{Leu(UUR)} (FIG. 2, panels A, C) which is responsible for more than 50% of human mt-tRNA mutation-associated diseases (MELAS, MIDD), and of cell models carrying the m·8344A>G mutation in the gene coding for mt-tRNA^{Lys}, which is associated with MERRF (FIG. 2, panels B, D), of the natural β32_33 peptide, and also
resulted, when exogenously administered up to 20 μM, safe in both mutant and wild-type cells (FIG. 3) and also

extremely stable in human plasma, since, in a first experiment, it did not undergo detectable degradation after 3 hours, as opposed to the β32_33 peptide that was degraded by 30% (FIG. 4, panel A) and, in a second experiment, more than 50% PMT it was present after 6 h, as opposed to the β32_33 peptide that was degraded by 90% (FIG. 4, panel A). These results also demonstrate that, as mentioned above, while peptide bonds between d-amino acids are often more stable in vivo with respect to peptide bonds between I-amino acids, this is not an absolute rule, since not all sources of in vivo degradation may be predicted a priori, therefore the superior in vivo PMT stability with respect to the β32_33 peptide could only be demonstrated by the plasma stability experiment.

In addition, surprisingly with respect to the results observed with the peptide of SEQ ID NO 4 reported in the state of the art, the conjugation with a mt-targeting sequence of SEQ ID NO 8 (also consisting of d-amino acids only) at the N terminus of the peptide of SEQ ID NO 1 (herein also indicated as PMT) of the invention, the inventors found that viability of m·3243A>G mutant cybrids was improved with the peptide of SEQ ID NO 5 (herein also indicated as M-PMT) at a 10-fold lower concentration with respect to the PMT peptide.

The same properties were shown by fragments of the peptide having SEQ ID NO 1, as shown in the figures.

Therefore, the peptides having SEQ ID NO 1 or 5, characterised in that they consist exclusively of d-amino acids as well as fragments of the same, have been shown as being excellent peptide-mimetics of the β32_33 peptide disclosed in Perli et al 2016 and Perli et al 2020 (SEQ ID NO 4), retaining the rescuing activity of the β32_33 peptide and showing enhanced relevant characteristics such as stability.

The present invention hence relates to a peptide having SEQ ID NO 1, where all the I-amino acids of the β32_33 peptide disclosed in Perli et al 2016 and Perli et al 2020 (SEQ ID NO 4), are substituted by d-amino acids, and fragments thereof, which surprisingly maintain rescuing activity toward mutations in mt-tRNAs and an enhanced stability in blood compared to the stability in blood of the peptide having SEQ ID NO 4, said peptide having SEQ ID NO 1 being optionally conjugated with an mt-targeting sequence consisting of d-amino acids.

Object of the present inventions are:

A peptide having SEQ ID NO 1 and/or fragments thereof of at least 8 amino acids of length, wherein said peptide entirely consists of d-amino acids, said peptide or fragments thereof being optionally conjugated at the N-terminus with an mt-targeting sequence; the peptide of SEQ ID NO 1 and/or fragments thereof as defined in the description and in the claims for use as a medicament, said peptide or fragments thereof being optionally conjugated at the N-terminus with an mt-targeting sequence;

the peptide of SEQ ID NO 1 and/or fragments thereof as defined in the description and in the claims, said peptide or fragments thereof being optionally conjugated at the N-terminus with an mt-targeting sequence, for use in the treatment of human mt-tRNA-related diseases and/or variants thereof as defined in the present specification;

a pharmaceutical composition comprising the peptide of SEQ ID NO 1 and/or fragments thereof as defined in the description and in the claims, said peptide or fragments thereof being optionally conjugated at the N-terminus with an mt-targeting sequence, and at least one pharmaceutically acceptable carrier;

said pharmaceutical composition for use as a medicament;

said pharmaceutical composition for use in the treatment of human mt-tRNA-related diseases;

a process for the preparation of the pharmaceutical composition according to the description and the claims comprising admixing one or more peptide of SEQ ID NO 1 and/or fragments thereof as defined above said peptide or fragments thereof being optionally conjugated at the N-terminus with an mt-targeting sequence, with at least one pharmaceutical acceptable carrier;

a method for the treatment of mt-tRNA-related diseases comprising administering to a subject in need thereof a therapeutically effective amount the peptide of SEQ ID NO 1 and/or fragments thereof said peptide or fragments thereof being optionally conjugated at the N-terminus with an mt-targeting sequence, or of the pharmaceutical composition as defined in the description and in the claims,

and the use the peptide of SEQ ID NO 1 and/or fragments thereof as defined in the description and in the claims said peptide or fragments thereof being optionally conjugated at the N-terminus with an mt-targeting sequence, or of the pharmaceutical composition as defined in the description and in the claims for the preparation of a medicament for the treatment of mt-tRNA-related diseases wherein one or more of said peptides is admixed at least with a pharmaceutically acceptable carrier thereby obtaining a pharmaceutical composition as defined in the description and in the claims.

### GLOSSARY

In the present description β32_33 peptide or Bp, indicates the peptide previously reported to have rescuing activity on mutant cells (Perli et al, FASEB J, 2020 and Perli et al” Hum mol genet 2016, Vol 25 No 5 903-915), herein also reported as peptide having SEQ ID NO 4.

The peptide having SEQ ID NO 1, which is a peptide-mimetic-therapeutic of the β32_33 peptide, is also indicated in the present description and figures as PMT.

The peptide having SEQ ID NO 5, which is a peptide-mimetic-therapeutic of the β32_33 peptide conjugated at N-terminus with a designed mt-targeting sequence having SEQ ID NO 8 is also indicated in the present description and figures as M-PMT.

The PMT fragment of the invention comprising PMT residues 1-8 having SEQ ID NO 2 is also indicated in the present description and figures as PMT-8a.

The PMT fragment of the invention comprising PMT residues 5-12 having SEQ ID NO 3 is also indicated in the present description and figures as PMT-8b.

The M-PMT fragment of the invention having SEQ ID NO 6, comprising PMT residues 1-8 having SEQ ID NO 2 conjugated at N-terminus with an mt-targeting sequence having SEQ ID NO 8 is also indicated in the present description and figures as M-PMT-8a.

The M-PMT fragment of the invention, having SEQ ID NO 7, comprising PMT residues 5-12 having SEQ ID NO 3 conjugated at N-terminus with an mt-targeting sequence having SEQ ID NO 8 is also indicated in the present description and figures as M-PMT-8b.

According to the present description and to the scientific literature, a mt-targeting or mt-penetrating sequence, is an N-terminal sequence that specifically directs and localises the protein/peptide to which it is bound, to the mitochondria, i.e. a mitochondrial transporter cell-permeable peptide (including a designed, not naturally occurring one) that is able to enter mitochondria or, in other words, a peptide that exhibits efficient cellular uptake, and specific mitochondrial localisation. In all parts of the description and of the claims SEQ ID NO 1, 2, 3, 5, 6 and 7 refers to a sequence consisting exclusively of d-amino acids.

According to the present description, the peptide of SEQ ID NO 1 or the fragments thereof having SEQ ID NO 2 or 3, whether conjugated at N-terminus with an mt-targeting sequence, or not conjugated with said mt-targeting sequence, are indicated also as “peptides”, however, peptides having SEQ ID NO 5, 6, and 7 can also be referred to as “conjugated peptides” due to the fact that they result from a conjugation at the N-terminus of the peptide of SEQ ID NO 1 or of one of its fragments according to the present description, with an mt-targeting sequence.

In the present description mt-tRNA-related diseases or syndromes have the meaning commonly intended in the art and refers to diseases or syndromes related to (caused by) mutations of mitochondrial tRNAs (mt-tRNAs), preferably point mutations of mitochondrial tRNAs.

In the present description mutations in mitochondrial genes (mt) coding for mt-tRNAs include m·3243A>G in MT-TL1 human gene encoding mt-tRNA^{Leu(UUR)}=>which causes MELAS and MIDD; m·8344A>G in MT-TK human gene encoding mt-tRNA^{Lys}=>MERRF, m·4277T>C mutation in the mt-tRNA^{IIe} (MTTI) gene causing hypertrophic cardiomyopathy and m·1630A>G mutation in the mitochondrial tRNA^{Val} (MTTV) causing mitochondrial encephalopathy, lactic acidosis and stroke-like episodes.

The abbreviation “mt” in the present description and claims, as well as in the pertaining state of the art, stands for “mitochondrial”.

As used herein, an “effective amount” is defined as the amount required to confer a therapeutic effect on the treated subject, and is typically determined based on age, surface area, weight, and condition of the subject.

### DETAILED DESCRIPTION OF THE DRAWINGS

FIG. 1. Upon exogenous administration to mutant cells, all the constructs reported in the image are able to penetrate cell membranes and colocalize with mitochondria.

Top row (constructs): exogenously administered constructs covalently linked to a fluorescent dye (Cy5) are localized within cells. This result indicates that all constructs are able to penetrate cell membranes.

Middle row (Mitotracker red): mitochondria within the same cells shown in the top row are highlighted by Mitotracker red, a dye able to link specifically and exclusively to mitochondria.

Bottom row (merge): co-localization of constructs and mitochondria is revealed by a filter setting for both dyes.

The cells used for the experiments are trans-mitochondrial hybrids (hereafter named cybrids) bearing the m·3243A>G mutation in mt-tRNA^{Leu(UUR)}, which is associated with the MELAS syndrome.

The constructs used for the experiments are: the β32_33 peptide (βp), previously reported to have rescuing activity on mutant cells (Perli et al, FASEB J, 2020 and Perli et al Hum mol genet 2016, Vol 25 No 5 903-915); the peptide-mimetic-therapeutic of SEQ ID NO 1 (PMT); the PMT fragments comprising PMT residues 1-8 of SEQ ID NO 2 (PMT-8a) and 5-12 of SEQ ID NO 3 (PMT-8b); and the β32_33 peptide linked to elamipretide (E), a different peptide previously reported by Sabbah H N et al 2016 to possess mitochondrial targeting properties and putative mitochondrial protective activity [Sabbah H N, Gupta R C, Kohli S, Wang M, Hachem S, Zhang K. Chronic Therapy With Elamipretide (MTP-131), a Novel Mitochondria-Targeting Peptide, Improves Left Ventricular and Mitochondrial Function in Dogs With Advanced Heart Failure. Circ Heart Fail. 2016 February;9 (2):e002206. doi: 10.1161/CIRCHEARTFAILURE.115.002206. PMID: 26839394; PMCID: PMC4743543.]. Names of all constructs are followed by “—C” to indicate that they are linked to Cy5.

Cells were incubated for 24 hours with 0.25 μM of constructs. Half an hour before imaging, cells were stained with Mitotracker Red. Finally, fluorescence signals were detected with a laser scanning confocal microscope. PCC: Pearson's Correlation Coefficient (mean±SEM of six images).

FIG. 2. Following exogenous administration, the PMT significantly improves cell viability and mitochondrial respiration of mutant cells.

Top row: Viability of compound-treated cells. The X and Y axes show the compounds used and the percentage of viable cells following treatment, respectively.

Bottom row: Oxygen consumption of compound-treated cells. The X and Y axes show the compounds used and the amount of consumed oxygen expressed in fMoles per minute per cell, respectively.

The first bar of each graph represents cells without a pathological phenotype, treated with vehicle only. WT: wild-type; I-8344: cells with extremely low levels of mutation m·8344A>G in mt-tRNA^{Lys}. The second bar of each graph represents cells with a pathological phenotype, bearing either the MELAS-causing m·3243A>G in mt-tRNA^{Leu(UUR)} or the MERRF-causing m·8344A>G in mt-tRNA^{Lys}, treated with vehicle only. All the other bars of each graph represent cells with a pathological phenotype, treated with different compounds. 3243: m·3243A>G mutant cells; H-8344: high m·8344A>G mutation load.

The cells used for the experiments are cybrids, as in FIG. 1.

The compounds used for the experiments are the same as those listed in FIG. 1 (i.e., βp; PMT; PMT-8a; PMT-8b; and E-βp) plus elamipretide (E). The E-βp peptide was used in order to verify whether the combination of the prior art peptide (SEQ ID NO 4) or of the peptide of SEQ ID NO 1 with elamipretide had a synergic effect as elamipretide has been described as a mitochondrial targeting sequence. The results of the experiments show that elamipretide does not provide additional advantageous effects to the tested peptides (SEQ ID NO 1 and SEQ ID NO 4) In this case compounds are not linked to Cy5, which is only used for fluorescence experiments. V indicates cells treated with an empty vehicle.

For viability assessment, cells were plated in either glucose or galactose medium. The reason for this is that a viability phenotype can be appreciated in cells growing on galactose, which forces cells to rely on mitochondrial respiration, but not in cells growing on glucose. After 24 hours incubation, the number of viable cells in the galactose medium was normalized to the number of viable cells in glucose at the same time point, which represents the normal growth condition. Data are compared with the value of mutant cells incubated with vehicle only. Mean±SEM of at least three independent experiments is shown.

Oxygen consumption rate was measured on cells grown on glucose, since variations in this parameter can be appreciated in cells growing in this medium, after 36 hours of treatment. Data are shown compared with the value of the vehicle mutant cells. Mean±SEM of three independent experiments is shown.

^{§}p<0.05, ^{§§§§}p<0.0001 for m·3243A>G vs WT cells; ⁰⁰p<0.01, ^{ooo}p<0.001 for H-8344 vs I-8344 cells; *p<0.05, **p<0.01, ***p<0.001 for cells incubated with compounds vs vehicle only.

FIG. 3. Exogenously administered PMT is neither cyto-or mito-toxic up to 20 μM in mutant and wild-type cells.

Images show the effect of increasing PMT concentrations on healthy and mutant cells, compared with the effect of a cytotoxic (C1) and a mitotoxic (C2) agent, evaluated using the Mitochondrial ToxGlo™ Assay.

The X and Y axis show the compounds incubated with cells and the ratio between fluorescent and luminescent signal, respectively. In this assay, fluorescence increase indicates a decrease in cell membrane integrity, and luminescence decrease indicates a decrease in cellular ATP levels. In each graph, the first bar represents the effect of cell treatment with the cytotoxic reagent digitonin (C1); this causes both an increase in fluorescence and a decrease in luminescence, which indicates cellular toxicity. The second bar represents the effect of cell treatment with the mitotoxic agent sodium azide (C2); this causes a decrease in luminescence and has no effect on fluorescence, which indicates mitochondrial toxicity. The other bars represent the effect of cell treatment with 5, 10 or 20 μM PMT; this has no effect on either fluorescence or luminescence, indicating absence of cyto-or mito-toxicicity. The horizontal black line represents fluorescence/luminescence signal ratio of untreated cells. WT: wild type; 3243: cells bearing the mutation m·3243A>G in mt-tRNA^{Leu(UUR)}; L-8344 and H-8344: cells bearing low and high load of mutation m·8344A>G in mt-tRNA^{Lys}.

Cells used for the experiments are cybrids, as in FIGS. 1 and 2. Compounds used are: PMT not linked to Cy5, at different concentrations; a cytotoxic agent (digitonin); and a mitotoxic agent (sodium azide).

Cybrids were plated on a 96-well plate in normal growth conditions (i.e., glucose medium) and treated with different PMT concentrations. In parallel, control cells (both wild type and mutated) were incubated with either 400 μg/ml digitonin (C1) or 100 μl sodium azide (C2) for three hours. Fluorescence and luminescence were measured with a GloMax Multi+Luminometer. Signals observed following each treatment (i.e., 5-10-20 μM PMT; C1; and C2) were normalized using values of untreated cells and expressed as fluorescence/luminescence ratio. Data are the mean±SEM of two independent experiments.

FIG. 4. The PMT, PMT-8a and PMT-8b undergo slower degradation than the β32_33 peptide in human plasma.

Panel A. Representative chromatographic profiles of β32_33 and PMT obtained after compound incubation for 3 h with human plasma from two healthy subjects. The X axis shows the time (in minutes) at which each compound is eluted by the chromatographic column. The Y axis indicates the signal intensity of the two peptides.

Panel B. Time course decay of β32_33, PMT, PMT-8a and PMT-8b incubated up to 72 h with human plasma from four healthy subjects. The X axis shows the time (in hours) at which the sample is analysed. The Y axis indicates the signal intensity of the four peptides. Samples of plasma were obtained from healthy volunteers and immediately used for the analysis. Either β32_33 peptide or PMT for the experiment in panel A, and β32_33 peptide, PMT, PMT-8a or PMT-8b for the experiment in panel B, was incubated in plasma at a final concentration of 0.2 mM, at 37° C., up to 3 h (A) or 72 h (B). At the indicated time points, i.e., T0 and 3 h (A) or T0, 1.5, 3, 6 and 72 h (B), aliquots of 200 μL were treated with 3 volumes of acetonitrile containing 1% formic acid and extracted by a solid phase extraction system to remove proteins and phospholipids. Samples were dried under vacuum, resuspended in 100 μL of 0.1% formic acid containing 5% acetonitrile, and analyzed with a Water Acquity H-Class UPLC system equipped with a single-quadruple mass detector with electrospray ionization source. Samples were separated onto a reverse phase C18 column by performing a gradient with two mobile phases consisting of 0.1% formic acid in water and 0.1% formic acid in acetonitrile at a flow rate of 0.5 mL/min. Quantification was performed by Selected lon Recording (SIR): at m/z=917.88, corresponding to the [M+2 H]²⁺ion obtained from either β32_33 or PMT; at m/z=961.42, corresponding to the [M+H]⁺ion obtained from PMT-8a; and at m/z=869.48, corresponding to the [M+H]⁺ion obtained from PMT-8b.

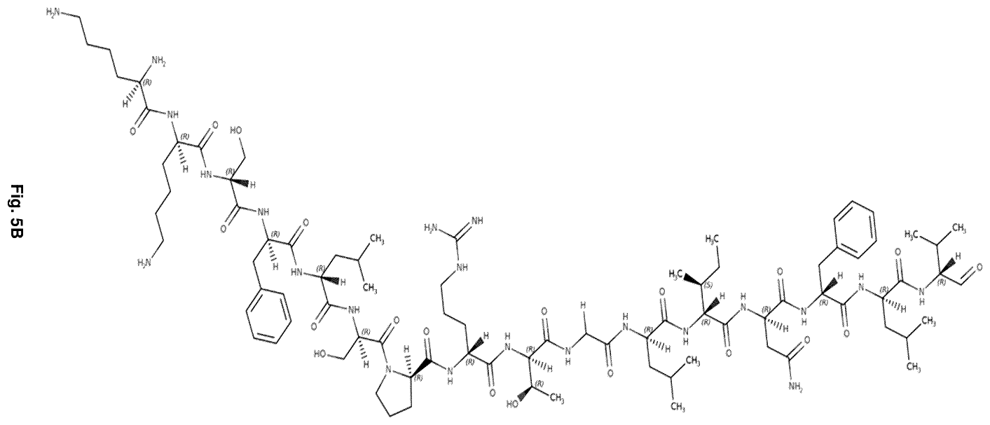

FIG. 5. Comparison between the structures of β32_33 peptide and PMT.

Chemical structure of the β32_33 peptide (A) and PMT (B, C). N, O and H atoms are explicitly indicated, whereas carbon atoms are implicit. Single and double chemical bonds lying on the plane are shown by single and double lines, respectively. Solid wedges indicate bonds projecting out towards the viewer. Broken wedges indicate groups receding away from the viewer. For each chiral center the S or R configuration is indicated. For each pair of corresponding amino acids (e.g., Lys1, Lys2, Ser3, ecc.) side-chains that are directed towards the viewer in the β32_33 peptide (A), are directed away from the viewer in the PMT (B, C), and side-chains that are directed away from the viewer in the β32_33 peptide (A), are directed towards the viewer in the PMT (A). In panel C, chemical groups of the PMT that have a different orientation with respect to the β32_33 peptide (A) are highlighted by grey ovals.

Since the relative position of all amino acid side-chains with respect to the main chain is opposite in the β32_33 peptide and in the PMT, interactions between the β32_33 peptide and target mt-tRNA involving both main chain and side chain atoms cannot be conserved in the PMT.

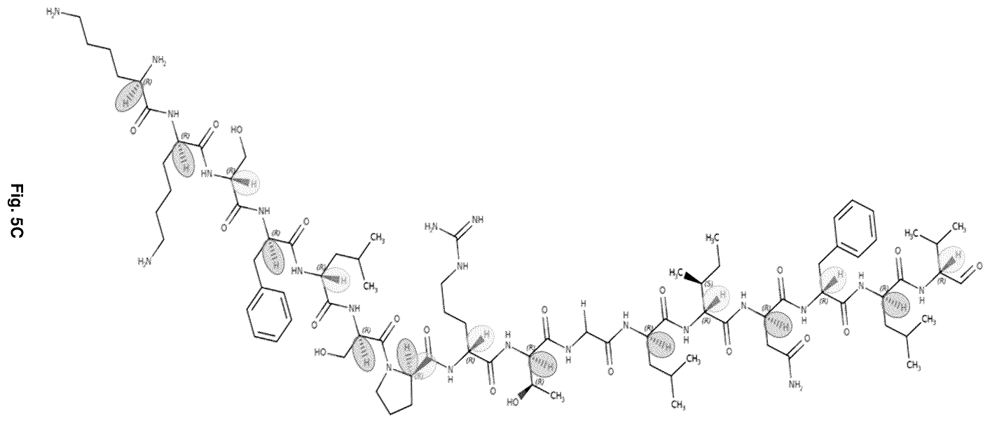

FIG. 6. Following exogenous administration, the M-PMT significantly improves viability of mutant cells at concentrations down to 0.5 μM.

Viability of compound-treated cells. The concentrations of the different compounds used for the experiments and the percentage of viable cells following treatment are shown in the X and Y axes, respectively. The first bar corresponds to wild type cells treated with vehicle only. The second bar represents cells bearing the MELAS-causing m·3243A>G in mt-tRNA^{Leu(UUR)}, treated with vehicle only. The additional bars show the effect of the PMT and M-PMT at decreasing concentrations on mutant cell viability. WT: wild-type cells. 3243: m·3243A>G mutant cells.

The cells used for the experiments are cybrids.

The compounds used for the experiment are: PMT at a 5, 2 and 0.5 μM concentration, and M-PMT at 5, 2 and 0.5 μM concentration. V indicates cells treated with an empty vehicle.

For viability assessment, cells were plated in either glucose or galactose medium. The reason for this is that a viability phenotype can be appreciated in cells growing on galactose, which forces cells to rely on mitochondrial respiration, but not in cells growing on glucose. After 24 hours incubation the number of viable cells in galactose medium was normalized to the number of viable cells in glucose (that represents the normal growth condition) at the same time point. Data are compared with the value of mutant cells incubated with vehicle only. Means±SEM of at least two independent experiments are shown.

^{ooo}p<0.0001 for m·3243A>G vs WT cells; *p<0.05 for cells incubated with compounds vs vehicle only.

### SEQUENCES DESCRIPTION

SEQ ID NO 1 PMT all amino acids are d-amino acids KKSFLSPRTALINFLV

SEQ ID NO 2 PMT-8a all amino acids are d-amino acids KKSFLSPR

SEQ ID NO 3 PMT-8b all amino acids are d-amino acids LSPRTALI

SEQ ID NO 4 β32_33 KKSFLSPRTALINFLV (Perli et al, FASEB J, 2020 and Perli et al Hum mol genet 2016, Vol 25 No 5 903-915) (all amino acids are I-amino acids)

SEQ ID NO 5 corresponds to SEQ ID NO 1 conjugated with mitochondrial targeting sequence FRFK, all amino acids are d-amino acids FRFKKKSFLSPRTALINFLV

SEQ ID NO 6 corresponds to SEQ ID NO 2 conjugated with mitochondrial targeting sequence FRFK, all amino acids are d-amino acids FRFKKKSFLSPR

SEQ ID NO 7 corresponds to SEQ ID NO 2 conjugated with mitochondrial targeting sequence FRFK, all amino acids are d-amino acids FRFKLSPRTALI

SEQ ID NO 8 artificial mitochondrial targeting/penetrating sequence 1 FRFK, all amino acids are d-amino acids

SEQ ID NO 9 artificial mitochondrial targeting/penetrating sequence 2 FRAₓK, all amino acids are d-amino acids

SEQ ID NO 10 mitochondrial targeting/penetrating sequence 3 Fd (R) FK, only R is a D amino acid, Horton K L et al, 2008

SEQ ID NO 11 artificial mitochondrial targeting/penetrating sequence 4 AₓRAₓK, all amino acids are d-amino acids

SEQ ID NO 12 artificial mitochondrial targeting/penetrating sequence 5 FRFKFRFK, all amino acids are d-amino acids

SEQ ID NO 13 artificial mitochondrial targeting/penetrating sequence 6 FRAₓKFRAₓK, all amino acids are d-amino acids

SEQ ID NO 14 artificial mitochondrial targeting/penetrating sequence 7 AₓRAₓKAₓRAₓK, all amino acids are d-amino acids

SEQ ID NO 15 artificial mitochondrial targeting/penetrating sequence 8 RKKRRQRRR, all amino acids are d-amino acids

SEQ ID NO 16 artificial mitochondrial targeting/penetrating sequence 9 FRF₂K, all amino acids are d-amino acids

SEQ ID NO 17 artificial mitochondrial targeting/penetrating sequence 10 FRY_{Me}K, all amino acids are d-amino acids

SEQ ID NO 18 artificial mitochondrial targeting/penetrating sequence 11 FRYK, all amino acids are d-amino acids

SEQ ID NO 19 artificial mitochondrial targeting/penetrating sequence 12 YRYK, all amino acids are d-amino acids

SEQ ID NO 20 mitochondrial targeting/penetrating sequence 13 Fd(R)AₓK, only R is a d amino acid, Horton K L et al, 2008

SEQ ID NO 21 mitochondrial targeting/penetrating sequence 14 Aₓd(R)AₓK, only R is a d amino acid, Horton K L et al, 2008

SEQ ID NO 22 mitochondrial targeting/penetrating sequence 15 Fd(R)FKFd(R)FK, only R is a d amino acid, Horton K L et al, 2008

SEQ ID NO 23 mitochondrial targeting/penetrating sequence 16 Fd(R)AₓKFd(R)AₓK, only R is a d amino acid, Horton K L et al, 2008

SEQ ID NO 24 mitochondrial targeting/penetrating sequence 17 Aₓd(R)AₓKAₓd(R)AₓK, only R is a d amino acid, Horton K L et al, 2008

SEQ ID NO 25 mitochondrial targeting/penetrating sequence 18 RKKRRQRRR, Horton K L et al, 2008

SEQ ID NO 26 mitochondrial targeting/penetrating sequence 19 Fd(R)F₂K, only R is a d amino acid, Horton K L et al, 2008

SEQ ID NO 27 mitochondrial targeting/penetrating sequence 20 Fd(R)Y_{Me}K, only R is a d amino acid, Horton K L et al, 2008

SEQ ID NO 28 mitochondrial targeting/penetrating sequence 21 Fd(R)YK, only R is a d amino acid, Horton K L et al, 2008

SEQ ID NO 29 mitochondrial targeting/penetrating sequence 22 Yd(R)YK, only R is a d amino acid, Horton K L et al, 2008

Abbreviations in the sequences above: F2: diphenylalanine; Aₓ: Cyclohexylalanine; YMe: methylated tyrosine. When the sole d-aminoacid is arginine, the aminoacid is indicated in the sequence as d(R).

### DETAILED DESCRIPTION OF THE INVENTION

As discussed in the summary of the invention, the peptide of the invention is a peptide-mimetic compound, hereafter indicated as “PMT”. The PMT comprises only d-amino acids (indicated by one-letter code preceded by lower-case “d” letter), the sequence of which is SEQ ID NO 1:

d(K)d(K)d(S)d(F)d(L)d(S)d(P)d(R)d(T)d(A)d(L)d(I)d(N)d(F)d(L)d(V).

As shown in the figures and discussed in the experimental part below, the PMT, as well as fragments of the same, is able to penetrate cell and mitochondrial membranes upon exogenous administration (FIG. 1), and to rescue the defective phenotype of cell models carrying mt-tRNA mutations (FIG. 2).

Additionally, exogenously administered PMT is safe up to 20 μM in both mutant and wild-type cells and finally, the PMT is extremely stable in human plasma, since after 3 hours incubation in this medium, in a first experiment the PMT is 100% available vs. 70% of the β32_33 peptide (FIG. 4A), and in a second experiment the PMT is>63% available vs. only 17% of the β32_33 peptide (FIG. 4B).

The invention relates to a peptide having SEQ ID NO 1, said peptide being characterised by consisting exclusively of d-amino acids and to fragments thereof, in particular fragments of at least 8 amino acids, as said peptide and fragments thereof of the indicated size have shown to be excellent peptide-mimetics of the β32_33 peptide (Perli et al, FASEB J, 2020 and Perli et al” Hum mol genet 2016, Vol 25 No 5 903-915) in terms of biological activity i.e. rescuing defective phenotype of cell models carrying mt-tRNA mutations, the mimetics showing the advantageous feature of being more stable in plasma than the natural peptide.

In an advantageous embodiment of the invention, said peptide having SEQ ID NO 1 and fragments thereof, in particular fragments of at least 8 amino acids, can be conjugated at the N-terminus with an mt-targeting sequence. FIG. 6 shows that conjugation with an mt-targeting sequence, such as SEQ ID NO 8, surprisingly improves the effectiveness (i.e., the rescuing activity) of the peptidomimetics of the invention of about 10 folds.

Hence, object of the invention are also peptides consisting of a d mt-targeting sequence conjugated at the N-terminus of the peptides having SEQ ID NO 1, SEQ ID NO 2 and SEQ ID NO 3.

In fact, contrary to the data disclosed in Perli et al 2020, wherein the use of a mt-targeting sequence did not increase the rescuing activity and mitochondrial localisation of the peptide having SEQ ID NO 4, the conjugation of the peptides of the invention with a mt-targeting sequence did dramatically increase the rescuing activity of the d-peptides of the invention.

Preferably, the mt-targeting sequence of the invention, is a sequence of 3-11 amino acids, preferably of 3 to 6 amino acids, and comprises at least one arginine and/or at least one lysine and/or at least one phenylalanine residue.

Preferably at least one of said arginine and/or at least one phenylalanine residues are d-arginine and/or d-lysine and/or d-phenylalanine.

According to the invention the mt-targeting sequence can be a sequence selected from SEQ ID NO 8 to SEQ ID NO 29. In an embodiment of the invention the fragments of the peptide of SEQ ID NO 1, conjugated at N-terminus with one of said mt-targeting sequence are the peptides of SEQ ID NO 2 or SEQ ID NO 3.

In a preferred embodiment, the mt-targeting sequence consists only of d-aminoacids, in a further preferred embodiment said mt-targeting sequence is selected from SEQ ID NO 8, SEQ ID NO 9, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or SEQ ID NO 19.

In a further preferred embodiment, said mt-targeting sequence consisting only of d-aminoacids is SEQ ID NO 8.

In a preferred embodiment the peptides conjugated at N-terminus with the mt-targeting sequence having SEQ ID NO 8, are the peptides having SEQ ID NO 5, SEQ ID NO 6 and SEQ ID NO 7.

Given the important rescuing activity shown by the peptide-mimetics herein disclosed, the invention also relates to the peptide having SEQ ID NO 1 and/or fragments thereof according to any of the embodiments disclosed, preferably conjugated at the N-terminus with an mt-targeting sequence according to any one of the embodiments disclosed above, for use as a medicament.

In an embodiment the invention also relates to variants of SEQ ID NOs 1, 2, 3, 5, 6 and 7 comprising one or more of the following chemical modifications:
modification/s of residues d-Phe 4, d-Leu 5, d-Arg 8 and/or d-Thr 9 of SEQ ID NO 1;
modification/s of residues d-Phe 8, d-Leu 9, d-Arg 12 and/or d-Thr 13 of SEQ ID NO 5;
modifications of the peptide bond between d-Phe 4 and d-Leu 5 of SEQ ID NO 1 or 2(in that this peptide bond is not present in the PMT-8b fragment, which does not undergo degradation at all in human plasma),
modifications of the peptide bond between d-Phe 8 and d-Leu 9 of SEQ ID NO 5 or 6 (see above);
modifications of the peptide bond between d-Arg 8 and d-Thr 9 of SEQ ID NO 1, d-Arg 4 and d-Thr 5 of SEQ ID NO 3, (in that this peptide bond is not present in the PMT-8a fragment, which does not undergo degradation at all in human plasma);
modifications of the peptide bond between d-Arg 12 and d-Thr 13 of SEQ ID NO 5, d-Arg 8 and d-Thr 9 of SEQ ID NO 7 (see above).

All the above modifications are aimed at improving PMT or fragments thereof (optionally conjugated with the mt-targeting sequence) as listed in table 1, plasma stability while retaining rescuing activity; or variants of the PMT comprising chemical modifications of additional residues the peptide bonds between which will be shown not to undergo degradation by the analysis of the PMT fragments resulting from incubation in human plasma aimed at improving PMT plasma stability while retaining rescuing activity. Also said variants can be preferably conjugated at the N-terminus with an mt-targeting sequence according to any of the embodiments disclosed above. Preferably said variants are conjugated with mt-targeting sequence of SEQ ID NO 8.

In particular, the invention relates to the peptide having SEQ ID NO 1 and/or fragments thereof, optionally conjugated at the N-terminus with an mt-targeting sequence according to any of the embodiments disclosed, for use in the treatment of mt-tRNA-related diseases.

As explained in the state of the art as well as in the summary of the invention and in the glossary, human mt-tRNA-related diseases are diseases caused by mutations, in particular point mutations of various mt-tRNA coding genes which result in mutations in the mt-tRNA itself.

Said diseases show a panel of different symptoms normally affecting highly oxygen consuming tissues such as brain, heart, muscles etc., i.e. tissues in which the role of mitochondria is extremely relevant. A non-limiting example of mt-tRNA-related diseases according to the invention includes mitochondrial myopathy, MERRF (Myoclonic Epilepsy with Ragged Red Fibers), MIDD (Maternally Inherited Diabetes and Deafness) and MELAS (mitochondrial encephalomyopathy, lactic acidosis, and stroke-like episodes).

In an embodiment of the invention, the mt-tRNA-related disease is caused by a point mutation in a gene encoding one of the following mitochondrial tRNAs mt-tRNA^{Leu(UUR)} mt-tRNA^{Lys} mt-tRNA^{IIe} and mt-tRNA^{Val}

In particular, mt-tRNA^{(Leu)(UUR)}, and mt-tRNA^{(Lys)}, which are responsible of about 85% of the mt-tRNA-related disease.

In an embodiment of the invention, the peptide having SEQ ID NO 1 and/or fragments thereof as defined herein, optionally conjugated at the N-terminus with an mt-targeting sequence according to any of the embodiments disclosed above, is in the treatment of mt-tRNA-related diseases, wherein said mt-tRNA-related disease is caused by a point mutation selected from m·3243A>G in the MT-TL1 human gene encoding mt-tRNA^{Leu(UUR)} or m·8344A>G in the MT-TK human gene encoding mt-tRNA^{Lys} or m·4277T>C mutation in the mt-tRNA^{IIe} in the human gene MT-TI or m·1630A>G mutation in mt-tRNA^{Val} in the human gene MT-TV.

When the disease is caused by one of the mutations indicated above, said disease is MIDD, MELAS or MERRF.

A further object of the present invention is a pharmaceutical composition comprising one or more peptide and/or fragments thereof as defined in any one of claims 1 to 5 and at least one pharmaceutically acceptable carrier.

Non limited examples of suitable pharmaceutical composition are for systemic, oral, injectable, aerosol, oropharyngeal, nasal administration.

The composition of the invention can be in the form of a solid, semi-solid, liquid, emulsion, gel, nebulizable product and the like.

The composition of the invention can also comprise one or more of the peptides having SEQ ID NO 1, 2 3, 5, 6 and/or 7, complexed in the form of nanovesicles, liposomes and nanoparticles, based on either inorganic compounds or proteins, including human ferritin and variants thereof.

The invention hence relates also to the pharmaceutical composition herein disclosed and claimed for use as a medicament, in particular for use in the treatment of mt-tRNA-related diseases.

A non-limiting example of mt-tRNA-related diseases according to the invention includes mitochondrial myopathy, MERRF (Myoclonic Epilepsy with Ragged Red Fibers), MIDD (Maternally Inherited Diabetes and Deafness)) and MELAS (mitochondrial encephalomyopathy, lactic acidosis, and stroke-like episodes).

In an embodiment of the invention, the mt-tRNA-related disease is caused by a point mutation in a gene encoding one of the following mt-tRNAs: mt-tRNA^{Leu(UUR)}, mt-tRNA^{Lys} mt-tRNA^{IIe} and mt-tRNA^{Val}. In an embodiment of the invention, the pharmaceutical composition as defined herein, is in the treatment of mt-tRNA-related diseases, wherein said mt-tRNA-related disease is caused by a point mutation is m·3243A>G in the MT-TL1 human gene encoding mt-tRNA^{Leu(UUR)} or m·8344A>G in the MT-TK human gene encoding mt-tRNA^{Lys} or m·4277T>C mutation in the mt-tRNA^{IIe} in the human gene MT-TI or m·1630A>G mutation in mt-tRNA^{Val} in the human gene MT-TV.

When the disease is caused by one of the mutations indicated above, said disease is MIDD, MELAS or MERRF.

The invention also relates to a process for the preparation of the pharmaceutical composition as defined above and in the claims comprising admixing one or more peptide having SEQ ID NO 1 and/or fragments thereof, optionally conjugated at the N-terminus with an mt-targeting sequence according to any one of the embodiments disclosed above, as defined in the description and in the claims with at least one pharmaceutical acceptable carrier. The peptide/s of the invention can be synthesized by any technique commonly used in the art for the preparation of d-peptides and it can be purified, with conventional techniques, to pharmaceutical grade. Once prepared and purified, the d-peptide/s of the invention are formulated in the corresponding pharmaceutical compositions according to well-known techniques in the field together with the conventional carrier/s, excipient/s and the like; see for example the volume “Remington's Pharmaceutical Sciences 15a Ed.”

The compositions of the present invention may additionally contain other compatible adjunct components conventionally found in pharmaceutical compositions, not recited above, at their art-established usage levels. Thus, for example, the compositions may contain additional compatible pharmaceutically-active materials for combination therapy or may contain materials useful in physically formulating various dosage forms of the present invention, such as excipients, preservatives, anti-oxidants, thickening agents, stabilizers and the like.

The invention also relates to the use of the peptide having SEQ ID NO 1 and/or fragments thereof, optionally conjugated at the N-terminus with an mt-targeting sequence according to any one of the embodiments disclosed above, as herein defined and claimed in in vitro methods of pharmaco-toxicological studies, e.g., for the detection of PMT off targets, for the assessment of tissue specific PMT effect, for the investigation of PMT activity on additional diseases.

By way of example, for the assessment of tissue specific PMT effect the peptide having SEQ ID NO 1 and/or one or more fragments thereof optionally conjugated at the N-terminus with an mt-targeting sequence according to any one of the embodiments disclosed above, is put in contact with specific tissue cells or tissues or organoids optionally bearing one or more mutation in mt-tRNA genes resulting in mutations in the corresponding mt-tRNAs that affect the phenotype of said cells, tissues or organoids, and their capability of rescuing the cellular, tissue, organoid abnormal phenotype caused by said mutation/s is assessed.

Alternatively, the PMT or fragments thereof can be tested on healthy cells, tissues or organoids in order to identify undesired off-target effects thereof vs. untreated controls or the PMT or fragments thereof can be tested in combination with other compounds in order to identify potentially therapeutically effective active principle combinations.

By “affect the phenotype” in the sentences above it is intended that said mutation/s cause an abnormal phenotype and can therefore be mutation/s causing mtRNA-related diseases.

“Rescuing” the abnormal phenotype can be a partial rescue (from a more severe to a less severe phenotype, i.e., with respect to control untreated samples) as well as a full rescue (from abnormal to normal phenotype i.e., with respect to control samples not bearing the mutation/s).

The peptide/s of the invention can also be used in vitro, as described above, in combination with one or more additional compound in order to identify compounds that can have a pharmacological effect on mtRNA-related diseases

Additionally, the invention relates to a method for the treatment of mt-tRNA-related diseases comprising administering to a subject in need thereof, a therapeutically effective amount of peptide having SEQ ID NO 1 and/or fragments thereof optionally conjugated at the N-terminus with an mt-targeting sequence according to any one of the embodiments disclosed above, as defined in the description and in the claims (which are peptidomimetics of the peptide known in the art having SEQ ID NO 4) or of the pharmaceutical composition as defined in the description and in the claims.

A non-limiting example of mt-tRNA-related diseases treatable with the method of the invention comprises mitochondrial myopathy, MERRF (Myoclonic Epilepsy with Ragged Red Fibers), MIDD (Maternally Inherited Diabetes and Deafness) and MELAS (mitochondrial encephalomyopathy, lactic acidosis, and stroke-like episodes).

In an embodiment of the invention, the mt-tRNA-related disease is caused by a point mutation in a gene encoding one of the following mt-tRNAs: mt-tRNA^{Leu(UUR)}, mt-tRNA^{Lys} mt-tRNA^{IIe} and mt-tRNA^{Val}, which are responsible of more than the 85% of human mt-tRNA-related diseases.

In an embodiment of the invention, the invention relates to the treatment of mt-tRNA-related diseases, wherein said mt-tRNA-related disease is caused by a point mutation, wherein said mutation is m·3243A>G in the MT-TL1 human gene encoding mt-tRNA^{Leu(UUR)} or m·8344A>G in the MT-TK human gene encoding mt-tRNA^{Lys} or m·4277T>C mutation in the mt-tRNA^{IIe} in the human gene MT-TI or m·1630A>G mutation in mt-tRNA^{Val} in the human gene MT-TV.

When the disease is caused by one of the two mutations indicated above, said disease is MIDD, MELAS or MERF.

A further object of the invention is the use of a peptide having SEQ ID NO 1 and/or fragments thereof optionally conjugated at the N-terminus with an mt-targeting sequence according to any one of the embodiments disclosed above, as defined in the description and in the claims for the preparation of a medicament for the treatment of mt-tRNA-related diseases wherein one or more of said peptide having SEQ ID NO 1 and/or fragments thereof as defined in the description and in the claims is admixed at least with a pharmaceutically acceptable carried thereby obtaining a pharmaceutical composition as defined in the description and in the claims.

As stated above, mt-targeting sequences consisting of d-aminoacids only are preferred. All cells used in the experiments reported below were obtained from patients that have given their free and informed consent to said use according to current legislation.

### EXAMPLES

### Materials and Methods

Peptide synthesis

All constructs were synthesized with purity>85% by Pepscan (Pepscan Presto, Lelystad, The Netherlands).

The compounds used for the study are listed in Table 1.

| | | | SEQ |
|---|---|---|---|
| | | | ID |
| Compounds | Peptide | Reference | NO |
| βp, here | KKSFLSPRTALINFLV | Perli et al, FASEB | 4 |
| named also- | | J, 2020 (I-amino | |
| β32_33 | | acids) | |
| PMT (d-βp) | d(K)d(K)d(S)d(F)d(L)d(S)d(P)d(R) | Unpublished | 1 |
| | d(T)d(A)d(L)d(I) | | |
| | d(N)d(F)d(L)d(V) | | |
| PMT-8a (d-βp- | d(K)d(K)d(S)d(F)d(L)d(S)d(P)d(R) | Unpublished | 2 |
| 8a) | | | |
| PMT-8b (d-βp- | d(L)d(S)d(P)d(R)d(T)d(A)d(L)d(I) | Unpublished | 3 |
| 8b) | | | |
| M-PMT (d-M- | d(F)d(R)d(F)d(K)d(K)d(K)d(S)d(F) | Unpublished | |
| βp) | d(L)d(S)d(P)d(R)d(T)d(A)d(L)d(I) | | |
| | d(N)d(F)d(L)d(V) | | |
| M-PMT-8a (d- | d(F)d(R)d(F)d(K)d(K)d(K)d(S)d(F) | Unpublished | 6 |
| M-βp-8a) | d(L)d(S)d(P)d(R) | | |
| M-PMT-8b (d- | d(F)d(R)d(F)d(K)d(L)d(S)d(P)d(R) | Unpublished | |
| βp-8b) | d(T)d(A)d(L)d(I) | | |
| mt-targeting | d(F)d(R)d(F)d(K) | Unpublished | 8 |
| sequence 1 | | | |
| mt-targeting | d(F)d(R)d(Aₓ)d(K) | Unpublished | |
| sequence 2 | | | |
| mt-targeting | Fd(R)FK | Horton KL et al, | 10 |
| sequence 3 | | 2008; | |
| mt-targeting | d(Aₓ)d(R)d(Aₓ)d(K) | Unpublished | 11 |
| sequence 4 | | | |
| mt-targeting | d(F)d(R)d(F)d(K)d(F)d(R) | Unpublished | 12 |
| sequence 5 | d(F)d(K) | | |
| mt-targeting | d(F)d(R)d(Aₓ)d(K)d(F)d(R) | Unpublished | 13 |
| sequence 6 | d(Aₓ)d(K) | | |
| mt-targeting | d(Aₓ)d(R)d(Aₓ)d(K)d(Aₓ)d(R)d(Aₓ) | Unpublished | 14 |
| sequence 7 | d(K) | | |
| mt-targeting | d(R)d(K)d(K)d(R)d(R)d(Q)d(R)d | Unpublished | 15 |
| sequence 8 | (R)d(R) | | |
| mt-targeting | d(F)d(R)d(F₂)d(K) | Unpublished | 16 |
| sequence 9 | | | |
| mt-targeting | d(F)d(R)d(Y_{Me})d(K) | Unpublished | 17 |
| sequence 10 | | | |
| mt-targeting | d(F)d(R)d(Y)d(K) | Unpublished | 18 |
| sequence 11 | | | |
| mt-targeting | d(Y)d(R)d(Y)d(K) | Unpublished | 19 |
| sequence 12 | | | |
| mt-targeting | Fd(R)AₓK | Horton KL et al, | 20 |
| sequence 13 | | 2008 | |
| mt-targeting | Aₓd(R)AₓK | Horton KL et al, | 21 |
| sequence 14 | | 2008; | |
| mt-targeting | Fd(R)FKFd(R)FK | Horton KL et al, | 22 |
| sequence 15 | | 2008 | |
| mt-targeting | Fd(R)AₓKFd(R)AₓK | Horton KL et al, | 23 |
| sequence 16 | | 2008; | |
| mt-targeting | Aₓd(R)AₓKAₓd(R)AₓK | Horton KL et al, | 24 |
| sequence 17 | | 2008 | |
| mt-targeting | RKKRRQRRR | Horton KL et al, | 25 |
| sequence 18 | | 2008 | |
| mt-targeting | Fd(R)F₂K | Horton KL et al, | 26 |
| sequence 19 | | 2008 | |
| mt-targeting | Fd(R)Y_{Me}K | Horton KL et al, | 27 |
| sequence 20 | | 2008 | |
| mt-targeting | Fd(R)YK | Horton KL et al, | 28 |
| sequence 21 | | 2008 | |
| mt-targeting | Yd(R)YK | Horton KL et al, | 29 |
| sequence 22 | | 2008 | |

| | | |
|---|---|---|
| Elamipretide | Sabbah HN et al | // |
| | 2016 | |
| Elamipretide-β32_33 | Unpublished | // |

Abbreviations: F2: diphenylalanine; Aₓ: Cyclohexylalanine; YMe: methylated tyrosine.

Cell lines

Previously established osteosarcoma derived (143B.TK-) cybrid cell lines from patients, which bear either the m·3243A>G mutation in mt-tRNA^{Leu(UUR)} or the m·8344A>G mutation in mt-tRNA^{Lys}, and controls were used (generous gift from Dr Valeria Tiranti and Dr Valerio Carelli). The pathological mt-tRNA^{Leu(UUR)} mutant had a mutation load>98%. The mt-tRNA^{Lys} mutant had a mutation load of either ˜80% (H-8344) or ˜0% (I-8344). The high mutation load mutant was pathological, whereas the low mutation level did not show any detectable phenotype [Perli et al, Hum Mol Genet, 2016].

Cell culture

Cybrid cells were cultured in Dulbecco's modified Eagle's medium (DMEM), supplemented with 4.5 g/l d-glucose, 10% foetal bovine serum (FBS), 2 mM I-glutamine, 50 μg/ML uridine, 100 U/mL penicillin, and 100 mg/mL streptomycin (referred to as glucose medium) in a humidified atmosphere of 95% air and 5% CO2 at 37° C. For cell viability experiments, cells were grown either in glucose medium or in glucose-free DMEM, supplemented with 5 mM galactose, 110 mg/mL sodium pyruvate, and 10% FBS (referred to as galactose medium). The reason for using the latter medium is that a pathological phenotype can be appreciated in cells growing on galactose, which forces cells to rely on mitochondrial respiration, but not in cells growing on glucose.

Fluorescence microscopy

Constructs made of compounds listed in Table 1 linked to the Cy5 fluorophore via maleimide cross-linker, were administered to sub-confluent cybrid cell cultures at 0.25 μM in glucose medium. About 24 hours after treatment with different constructs, cells were incubated with 200 nM Mitotracker Red FM (LifeTechnologies Italia, Monza, Italy) for 30 minutes at 37° C. Subsequently, cells were visualized by confocal microscopy. Images of 800×800 px (at 88 nm/px) were acquired at the Olympus iX83 FluoView1200 laser scanning confocal microscope using a 60×NA1,2 water objective (Olympus Italia SRL Milano, Italy), zoom 3×, 559 nm, and 635 nm lasers and filter setting for MitoTracker Red and Cy5. The fluorescence images were analyzed with the ImageJ software (14, https://imagej.nih.gov/ij/, 1997-2018) to determine the Pearson's correlation coefficient.

Cell viability

To test the growth capability, cells were harvested and seeded at 30×10⁴ in 60 mm dishes in glucose medium for 24 hours with the addition of one of the compounds (each at 5 μM concentration). Cells were switched in glucose or galactose and after 24 hours cell viability was measured by the Trypan blue dye exclusion assay. Cells were harvested with 0.25% trypsin and 0.2% EDTA, washed, suspended in PBS in the presence of Trypan blue solution (Sigma-Aldrich) at 1:1 ratio and counted using a hemocytometer. The number of viable cells in galactose medium was expressed as a percentage of the number of cells in glucose medium.

Respirometry assay

Oxygen consumption rate (OCR) of cybrids incubated with compounds was evaluated with Clark type oxygen electrode (Hansatech Instruments, Norfolk, UK). After incubation with compounds, both control and mutant cybrids were maintained in glucose medium for 36 hours, then OCR was measured in intact cells (3×10⁶) in 1 mL DMEM lacking glucose supplemented with 10% sodium pyruvate.

Mitochondrial toxicity

Mitochondrial toxicity exerted by PMT was measured using the Mitochondrial ToxGlo™ Assay (Promega Italia Srl., Milano, Italy) according to the manufacturer's protocol. Cybrids were plated on a 96-well plate and treated with different concentrations of PMT (5, 10 and 20 μM). Twenty-four hours after treatment, control cells (both wild type and mutated) were incubated with either 400 ug/ml digitonin (a cytotoxic agent) or 100 μl sodium azide (mitotoxic agent) for three hours, as positive control for cyto-or mito-toxicity, respectively. Subsequently, cells were incubated with specific reagents and fluorescence or luminescence were measured with a GloMax Multi+Luminometer (Promega Italia Srl., Milano, Italy).

Statistical analysis

All data are expressed as mean±SEM. Data were analyzed by standard ANOVA procedures followed by multiple pair-wise comparison adjusted with Bonferroni corrections. Significance was considered at<0.05. Numerical estimates were obtained with Graphpad Prism 7 version (Graphpad Inc San Diego, CA, USA).

Plasma stability

In order to evaluate whether compounds (βp and d-βp) are stable in blood or hydrolyzed by plasma peptidases, we set up a chromatographic assay to evaluate compound concentration after incubation in human plasma.

Blood was drawn by venipuncture in vacutainer containing EDTA as an anticoagulant.

Two different samples from healthy volunteers were used. Plasma was separated by centrifugation and immediately used for the experiments. Each compound was dissolved in 500 uL plasma at a final concentration of 0.2 mM, and split into two aliquots, one of which was immediately analyzed to assess the basal compound level; the other was incubated at 37° C. for 3 hours under gentle shaking. In order to perform chromatographic analysis the samples were treated with 3 volumes of acetonitrile containing 1% formic acid, and then extracted by using the Ostro™ pass-through sample preparation system to remove proteins and phospholipids. Samples were dried under vacuum and then resuspended in 100 uL of 0.1% formic acid containing 5% acetonitrile, then directly injected onto the chromatographic column.

Chromatographic analyses were performed on a Water Acquity H-Class UPLC system (Waters, Milford, MA, USA), including a quaternary solvent manager (QSM), a sample manager with a flow through needle system (FTN), a photodiode array detector (PDA) and a single-quadruple mass detector with electrospray ionization source (ACQUITY QDa). Analyses were performed on a reverse phase C18 column (75 mm×3.2 mm i.d., 2.5 μm particle size). The mobile phase was solvent A, 0.1% formic acid in water, and solvent B, 0.1% formic acid in acetonitrile. The flow rate was 0.5 mL/min, the column temperature was set at 25° C. and the elution was performed by linearly increasing the concentration of solvent B up to 70% in 7 minutes. Mass spectrometric detection was performed in the positive electrospray ionization mode, using nitrogen as the nebulizer gas. Analyses were performed in the Total lon Current (TIC) mode with a mass range of 100-1200 m/z. The capillary voltage was 0.8 kV, cone voltage 8 V, ion source temperature 120° C. and probe temperature 600° C. Quantification of each compound was performed by Selected lon Recording (SIR) at m/z 917.88, corresponding to the

### RESULTS

### PMT and PMT Fragments (PMT-8a and PMT-8b) Penetrate Cell Membranes and Co-Localize with Mitochondria

The uptake and localization of Cy5-conjugated constructs in cybrids was assessed by flow cytometry, confocal microscopy, and immunoblot analysis on isolated mitochondria (FIG. 1). Confocal microscopy was performed using a specific live cell staining for mitochondria (Mitotracker FM Red). After 12 hours the fluorescent signal of all constructs was clearly detectable within cybrids. All constructs showed cellular uptake and a clear overlap with mt reticulum, as demonstrated by Pearson's correlation coefficients reflecting the mitochondrial specificity (FIG. 1). These results indicate that, upon exogenous administration to mutant cells, all of the constructs reported in the image are able to penetrate cell membranes and colocalize with mitochondria.

### Effect of PMT and PMT Fragments (PMT-8a and PMT-8b) on the Viability of m·3243A>G mt-tRNA^{Leu(UUR)} and m·8344A>G mt-tRNA^{Lys} Mutant Cybrids.

To evaluate the effect of the compounds on viability, cybrids were grown in glucose-free medium supplemented with galactose (galactose medium), a condition that both forces cells to rely on the mt respiratory chain for ATP synthesis and causes a significant growth reduction in the presence of mutations.

We observed that the PMT was able to induce a significant improvement of cell viability and apoptotic rate in both m·3243A>G and m·8344A>G mutant cybrids, as compared with non treated mutant cells (FIG. 2, top panels). The rescuing activity of PMT was comparable to that of the β32_33 peptide. The PMT-8b also significantly improved cell viability and apoptotic rate in m·3243A>G mutant.

### Effect of PMT and PMT Fragments (PMT-8a and PMT-8b) on Oxygen Consumption of m·3243A>G mt-tRNA^{Leu(UUR)} and m·8344A>G mt-tRNA^{Lys} mutant cybrids

To investigate whether increased cell viability was related to improved mt bioenergetics, we analyzed the respiratory capability of mutant and control cells by using the Clark type electrode. We demonstrated that the PMT determined a significant increase of oxygen consumption rate in both pathological mutants (FIG. 2, bottom panels). This activity is comparable to that of the β32_33 peptide in m·3243A>G mutant cybrids, and even higher than that of the β32_33 peptide in m·8344A>G mutant cybrids.

### Lack of Cyto-and Mitotoxicity of PMT and PMT Fragments (PMT-8a and PMT-8b)

We performed the Mitochondrial ToxGlo™ Assay to evaluate toxicity of increasing concentrations of exogenously administered PMT and PMT fragments. The PMT, PMT-8a and PMT-8b fragments resulted to be neither cytotoxic nor mitotoxic up to 20 μM in m·3243A>G mt-tRNA^{Leu(UUR)} mutant cybrids, m·8344A>G mt-tRNA^{Lys} mutant cybrids and healthy control cells.

### The PMT has Higher Stability than the β32_33 Peptide in Human Plasma.

To assess their plasma stability, two experiments were performed. In a first experiment, the β32_33 peptide or PMT was incubated in plasma samples from two healthy volunteers and the amount of each compound was measured before (T0) and after 3 hours plasma incubation (3 h). As shown in FIG. 4A, after 3 hours plasma incubation the PMT does not undergo visible degradation whereas only 70% of the β32_33 peptide is still available. In a second experiment, the β32_33 peptide, PMT, PMT-8a or PMT-8b was incubated in plasma samples from four healthy volunteers and the amount of each compound was measured before (T0) and at different time points after plasma incubation (i.e., 1.5, 3, 6 and 72 h). As shown in FIG. 4B, the PMT has higher plasma stability than the β32_33 peptide at all time points, although, at variance with the previous experiments, it did undergo detectable degradation. Conversely, after 72 h, the PMT-8a did not undergo detectable degradation, and 80% of the PMT-8b fragment was present, suggesting that the eight C-terminal residues of the PMT, which are not present in the PMT-8a and only four of which are present in the PMT-8b, and the character of which is mostly hydrophobic (e.g., d-Ala 10, d-Leu 11, d-IIe 12, d-Phe 14, d-Leu 15 and d-Val 16) may be at least partially responsible for peptide sequestration by plasma proteins endowed with hydrophobic pockets, such as serum albumin.

### M-PMT Ameliorates Viability of m·3243A>G mt-tRNA^{Leu(UUR)} at 10-Fold Lower Concentration with Respect to PMT.

To evaluate the effect of PMT and M-PMT on viability, cybrids were grown in glucose-free medium supplemented with galactose (galactose medium), a condition that both forces cells to rely on the mt respiratory chain for ATP synthesis and causes a significant growth reduction in the presence of mutations.

The M-PMT significantly improved viability of m·3243A>G mutant cybrids, as compared with non-treated mutant cells, at 5, 2 and 0.5 μM concentration (FIG. 6), whereas the PMT peptide exerted rescuing activity at 5 μM but not 2 and 0.5 μM concentration.

1. A peptide having the amino acid sequence SEQ ID NO 1 and/or a fragment thereof of at least 8 amino acids of length, or variants of said peptide or fragment, wherein said peptide entirely consists of d-amino acids; optionally further conjugated at the N-terminus with a mitochondrial (mt)-targeting sequence.

2. The peptide fragment according to claim 1 wherein said peptide fragments has the amino acid sequence SEQ ID NO 2 or SEQ ID NO 3.

3. The peptide and/or fragment thereof according to claim 1 further conjugated at the N-terminus with said mt-targeting sequence.

4. The peptide and/or fragment thereof according to claim 3 wherein said mt-targeting sequence is 3 to 11 amino acids.

5. The peptide and/or fragment thereof according to claim 4 wherein said mt-targeting sequence comprises at least one arginine and/or lysine and/or phenylalanine residue.

6. The peptide and/or fragment thereof according to claim 4 wherein said mt-targeting sequence is selected from the group consisting of SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 18 and SEQ ID NO 29.

7. The peptide and/or fragment thereof according to claim 3 wherein said mt-targeting sequence entirely consists of d-amino acids.

8. The peptide and/or fragment thereof according to claim 7 wherein said mt-targeting sequence is selected from the group consisting of SEQ ID NO 8, SEQ ID NO 9, SEQ ID NO 11, SEQ ID NO 12, SEQ ID NO 13, SEQ ID NO 14, SEQ ID NO 15, SEQ ID NO 16, SEQ ID NO 17, SEQ ID NO 18 or and SEQ ID NO 19.

9. The peptide according to claim 3 wherein said peptide has the amino acid sequence SEQ ID NO 5.

10. The peptide fragment according to claim 3 wherein said peptide fragments has the amino acid sequence SEQ ID NO 6 or SEQ ID NO 7.

11. (canceled)

12. A method for treating a mt-tRNA-related disease comprising administering the peptide and/or fragment thereof for use according to claim 1.

13. The method according to claim 12 wherein said mt-tRNA-related diseases is selected from the group consisting of mitochondrial myopathy, MERRF (Myoclonic Epilepsy with Ragged Red Fibers), MIDD (Maternally Inherited Diabetes and Deafness) and MELAS (mitochondrial encephalomyopathy, lactic acidosis, and stroke-like episodes).

14. The method according to claim 12 wherein said mt-tRNA-related disease is caused by a point mutation in a gene encoding one of the following mt-tRNAs: mt-tRNA^{Leu(UUR)}, mt-tRNA^{Lys}, mt-tRNA^{IIe} or mt-tRNA^{Val}

15. The method according to claim 14 wherein said mutation is m·3243A>G in the MT-TL1 human gene encoding mt-tRNA^{Leu(UUR)} or m·8344A>G in the MT-TK human gene encoding mt-tRNA^{Lys} or m·4277T>C mutation in the mt-tRNA^{IIe} in the human gene MT-TI or m·1630A>G mutation in mt-tRNA^{Val} in the human gene MT-TV.

16. (canceled)

17. A pharmaceutical composition comprising one or more peptides and/or fragments thereof as defined in claim 1 and at least one pharmaceutically acceptable carrier.

18. (canceled)

19. A method for treating a mt-tRNA-related disease comprising administering the pharmaceutical composition according to claim 17.

20. The method according to claim 19 wherein said mt-tRNA-related diseases is selected from the group consisting of mitochondrial myopathy, MERRF (Myoclonic Epilepsy with Ragged Red Fibers), MIDD (Maternally Inherited Diabetes and Deafness) and MELAS (mitochondrial encephalomyopathy, lactic acidosis, and stroke-like episodes).

21. The method according to claim 19 wherein said mt-tRNA-related disease is caused by a point mutation in a gene encoding one of the following mt-tRNAs: mt-tRNA^{Leu(UUR)}, mt-tRNA^{Lys}, mt-tRNA^{IIe} or mt-tRNA^{Val}.

22. The method according to claim 21 wherein said mutation is m·3243A>G in the MT-TL1 human gene encoding mt-tRNA^{Leu(UUR)} or m·8344A>G in the MT-TK human gene encoding mt-tRNA^{Lys} or m·4277T>C mutation in the mt-tRNA^{IIe} in the human gene MT-TI or m·1630A>G mutation in mt-tRNA^{Val} in the human gene MT-TV.

23. (canceled)

24. A process for preparation of a pharmaceutical composition comprising admixing one or more peptides and/or fragments thereof as defined in claim 1 with at least one pharmaceutical acceptable carrier.

25. (canceled)
